(19) United States
(12) Patent Application Publication
(10) Pub. No.: US 2005/0282895 A1
(43) Pub. Date: Dec. 22, 2005

(54) Antimicrobial compositions and methods of use thereof

(76) Inventors: Michael H. Dosch, Toronto (CA); Kurt Ostermann, Scarborough (CA)

Correspondence Address: MCHALE & SLAVIN, P.A., 2855 PGA BLVD, PALM BEACH GARDENS, FL, 33410, US

(21) Appl. No.: 10/873,040

(22) Filed: Jun. 21, 2004

(51) Int. Cl. A61K 31/195; A61K 31/19

(52) U.S. Cl. 514/557; 514/566; 514/574

(57) ABSTRACT

The present invention is directed to compositions which are useful for reducing and/or preventing bacterial infection/superinfection. The compositions include at least two weak organic acids in an inert carrier vehicle and are delivered at a pH of about 2.5 to 4.5. The weak organic acids are preferably acetic acid, vinegar, citric acid or combinations thereof. The inert carrier vehicle is preferably an aqueous based carrier in a gel form utilizing CARBOPOL as a gelling agent. The compositions of the invention are particularly applicable for reducing and/or preventing bacterial infection/superinfection of wounds.

### FIELD OF THE INVENTION

The instant invention relates generally to substances having antimicrobial activity and methods for their use; particularly to compositions useful for reducing wound infection and most particularly to compositions including a mixture of organic acids and EDTA in an inert carrier vehicle evidencing efficacy in reducing and preventing bacterial infection/superinfection of wounds.

### BACKGROUND OF THE INVENTION

Control of microbial growth in order to prevent disease, reduce infection and reduce contamination of food and water supplies has been a challenge since almost the beginning of time. Exposure to high temperatures and/or radiation is a known means for reduction of microbial contamination of surfaces. Additionally, numerous substances provide antimicrobial efficacy, including alcohol, bleach, acid, peroxide and vinegar to name but a few.

The use of vinegar as an antimicrobial agent is as old as the use of alcohol, and by around 1000 AD, hand washing with vinegar to avoid infection during autopsies was recommended in ancient medical texts from Chinese and Arabic sources (Chan et al. American Journal of Nephrology 14(4-6:295-301 1994). During the Middle Ages doctors attempted to protect themselves from contracting the “Black Death” (bubonic plague) with vinegar mixtures (Fradin et al. Medicine: Yesterday, Today and Tomorrow, Children's Press, Chicago 1989). Presently vinegar remains an additive in numerous antiseptic compositions.

Vinegar is an impure organic acid (acetic acid) and a rich source of many volatile contaminants (De Vincenzi et al. Food Additives and Contaminants 4(2) :161-218 1987). Vinegar is approved for human non-dietary use and is marginally effective as the main ingredient of vaginal douches although the mechanism is uncertain (Nyirjesy et al. Obstetrics and Gynecology 90(1):50-53 1997). The effectiveness of pure acetic acid, sodium acetate and vinegar have rarely been compared (Brighenti et al. European Journal of Clinical Nutrition 49(4) :242-247 1995) and never with respect to antimicrobial activity. Although sodium acetate is used in some vaginal douches, its effectiveness has not been measured (Chvapil et al. Obstetrics and Gynecology 52(1):88-93 1978). While acid sensitivity of bacteria is one element of antimicrobial activity, this is insufficient to explain the antimicrobial effects of vinegar, since some common food-borne bacteria are highly sensitive to vinegar, yet they survive gastric acid exposure and cause common intestinal disease (Nishikawa et al. International Journal of Food Microbiology 18(4):271-278 1993).

There is a limited amount of scientific literature on the possible antimicrobial effects of vinegar (Larghi et al. Rev Asoc Argentina Microbiology 7(3):86-90 1975 (Spanish); Fasanella RM Ophthalmalic Surgery 22(2):117-118 1991; Karapinar et al. International Journal of Food Microbiology 16(3):261-264 1992). Some of these observations have found their way into the food processing industry (Dickens et al. Poultry Science 73(4):576-581 1994; Entani et al. Kansenshogaku Zasshi 71(5):443-450 (Japanese) 1997). While vinegar treatment of chicken carcasses prior to freezing was shown to reduce bacterial contamination, there are no published reports of the effects of bacteria on living skin (Dickens et al. Poultry Science 73(4):576-581 1994). A widely employed skin application of vinegar occurs in the Pacific rim countries where, acting as a nematocyst inhibitor, vinegar is the recommended first aid treatment against potentially life threatening jelly fish stings (Fenner et al. Medical Journal of Australia 143(12-13):550-551, 554-555 1985; Beadnell et al. Medical Journal of Australia 156(9):655-658 1992; Fenner et al. Medical Journal of Australia 158(7):498-501 1993). In vitro tissue culture studies have suggested that acetic acid is toxic to human fibroblasts and keratinocytes in concentrations above 0.025%. This data contrasts with the frequent use of vinegar douches, which are well tolerated over prolonged periods of intravaginal use (Nyirjesy et al. Obstetrics and Gynecology 90(1):50-53 1997).

In addition to vinegar, the use of citric acid as a potential antimicrobial is practiced, however there is little published scientific literature to indicate its antimicrobial efficacy. Nevertheless, citric acid is a common preservative in foodstuffs and beverages such as soft drinks. It has also been used for antiseptic purposes in medical procedures, such as dental surgeries (Smith et al. American Association of Endodontists (AAE), Volume 12, number 2; abstract accessed from the AAE web site on Jun. 4, 2004).

Although there are a great number of methods and substances useful for providing antimicrobial activity; the majority of such methods and means are impractical for use in all situations, have limited effectiveness, are toxic and/or create microbial resistance. Thus, considering that microbial infestation is a persistent problem, there is a continuous need for novel antimicrobial methods and substances.

### DESCRIPTION OF THE PRIOR ART

Evaluation of the prior art reveals a plethora of antimicrobial compositions and methods. Several examples are noted herein.

U.S. Pat. No. 4,308,293 discloses compositions including pyroligneous acid and pyroligneous acid complexes useful as anti-fungal, antibacterial preservative agents for animal feedstuffs.

U.S. Pat. No. 6,110,908 discloses compositions including antimicrobial alcohols, antimicrobial lipids and combinations thereof useful as topical antiseptics.

U.S. Pat. No. 6,106,773 discloses an aqueous iodinated matrix material useful for disinfecting biological fluids.

U.S. Pat. No. 5,308,611 discloses chlorhexidine containing compositions useful as antiseptic agents.

U.S. Pat. No. 5,336,432 discloses a microemulsion gel having antiseptic and bleaching properties which is prepared from the combination of a water phase comprising water and propylene glycol with an oil phase generally comprising at least one surfactant, an emollient and an oil. Hydrogen peroxide is also added to the microemulsion.

U.S. Pat. No. 4,035,483 discloses an antiseptic composition comprising the reaction product of hypochlorite and a protein.

U.S. Pat. No. 5,785,972 discloses an antiseptic solution containing colloidal silver, helichrysum angusifolium or helichrysum italicum oil and raw honey emulsified with water soluble lecithin.

U.S. Pat. No. 5,855,922 discloses antiseptic compositions containing metal chlorite useful for treatment of dermal disorders.

U.S. Pat. No. 6,589,513 discloses a composition including cayenne and other natural ingredients useful as an oral antiseptic. Cayenne exhibits a synergistic effect on the actions of the other components of the composition.

Laufman et al. (American Journal of Surgery 157:359 1989) offers a review of dermal antiseptics.

However, despite the number of known antimicrobial agents, a need remains for more effective agents which are easily applicable in multiple situations. The instant invention provides a composition including a mixture of weak organic acids and EDTA having an antibacterial effect which is not dependent on H⁺ concentration (pH). This composition can be removed from the affected area after 10-20 minutes and will still provide protection/wound cleansing effect. This is distinct from current antimicrobial agents which require continuous presence at the affected area for extended periods of time. The prior art fails to disclose a composition with such characteristics. Thus, the composition of the instant invention satisfies a long felt need for antimicrobial agents with increased effectiveness.

While not wishing to be bound to any particular mode of operation, it is theorized that the combination of weak organic acids in the presence of EDTA exhibits a synergistic effect in denaturing microbial proteins.

### SUMMARY OF THE INVENTION

U.S. Pat. No. 6,469,066 B1 (Oct. 22, 2002), the contents of which are herein incorporated by reference, was issued to the present inventors as directed to compositions and their method and environment of use; wherein such compositions include a mixture of weak organic acids and EDTA in a gel-like carrier and demonstrate usefulness for targeting nociceptors, in particular TRPV-1 (transient receptor potential vanilloid) and ameliorating the sensation of pain, particularly in burn injuries. During application of these compositions it was discovered that these same compositions show surprising efficacy as bactericidal agents of broad target range. The present specification discloses methods for using these compositions for reducing and preventing bacterial infection/superinfection. The composition is particularly suitable for use in combat zones where traumatic injury is common and evacuation of casualties is often delayed.

The instant application is also related to U.S. application Ser. No. 10/232,080, filed Aug. 30, 2002, the contents of which are herein incorporated by reference.

The composition of the instant invention contains at least two weak organic acids blended according to specific process parameters within a liquid or a gel-like carrier, preferred, but non-limiting examples of such weak organic acids are vinegar, citric acid and acetic acid. Bactericidal efficacy was observed whether this composition was applied immediately upon contamination or after a delay. The composition may be applied in a gel form or spray form. It is also within the scope of the instant invention to form various articles which demonstrate the efficacious properties of the composition manifested in the form of a wound dressing, an absorbent bandage, a feminine hygiene product, a diaper or similar articles.

Accordingly, it is an objective of the instant invention to provide compositions which are effective for reducing and/or preventing bacterial infection/superinfection, particularly of wounds.

It is a further objective of the instant invention to provide a composition for reducing and/or preventing bacterial infection/superinfection comprising a therapeutically effective amount of at least two weak organic acids and EDTA.

It is yet another objective of the instant invention to select the therapeutically effective weak organic acids from a group including, but not limited to, acetic acid, vinegar, citric acid or combinations thereof, in a pharmacologically effective carrier, wherein the pH of the composition ranges from about 2.5 to 4.5.

It is yet another objective of the instant invention to provide a pharmacologically effective carrier for the weak organic acids which is aqueous based, preferably a gel form, utilizing a CARBOPOL gelling agent or equivalent.

It is yet another objective of the instant invention to provide a method for reducing and/or preventing bacterial infection/superinfection of a wound comprising applying to an affected area a composition including a therapeutically effective amount of at least two weak organic acids selected from the group including, but not limited to, acetic acid, vinegar, citric acid and combinations thereof in a pharmacologically effective carrier, in the presence of EDTA, wherein the pH of said composition ranges from approximately 2.5 to 4.5; and wherein said therapeutically effective amount provides reduction and/or prevention of bacterial infection/superinfection of said wound.

It is another objective of the instant invention to apply the composition disclosed herein as soon as possible after exposure of the wound to the external environment.

It is yet another objective of the instant invention to remove the composition disclosed herein from the affected area within a pre-determined period of time, preferably, but not limited to, removal between 5 and 20 minutes after application.

It is yet another objective of the instant invention to provide a kit for reducing and/or preventing bacterial infection/superinfection comprising a composition for reducing and/or preventing bacterial infection/superinfection of a wound including a therapeutically effective amount of at least two weak organic acids selected from the group consisting of acetic acid, vinegar, citric acid and combinations thereof in a pharmacologically effective carrier in the presence of EDTA, wherein the pH of said composition ranges from about 2.5 to 4.5; wrapping or bandage materials selected from the group consisting of wound dressings, absorbent bandages, feminine hygiene products, and diapers; and instructions for use.

Other objectives and advantages of the instant invention will become apparent from the following description taken in conjunction with the accompanying drawings wherein are set forth, by way of illustration and example, certain embodiments of the instant invention. The drawings constitute a part of this specification and include exemplary embodiments of the present invention and illustrate various objects and features thereof.

### ABBREVIATIONS AND DEFINITIONS

The following list defines terms, phrases and abbreviations used throughout the instant specification. Although the terms, phrases and abbreviations are listed in the singular tense the definitions are intended to encompass all grammatical forms.

As used herein, the term “microbial” means “of or related to microorganisms”.

As used herein, the term “microorganism” refers to any organism that can be seen only with the aid of a microscope. The compositions of the instant invention are particularly effective against both gram-positive and gram-negative bacteria. The terms “microorganism” and “microbe” are used interchangeably herein.

As used herein, the term “gram-positive bacteria” refers to bacterial cells which stain violet (positive) in the Gram stain assay. The Gram stain binds peptidoglycan which is abundant in the cell wall of gram-positive bacteria. In contrast, the cell wall of “gram-negative bacteria” is low in peptidoglycan, thus gram-negative bacteria adopt the counterstain in the gram stain assay.

As used herein, the term “bacterial contamination” is applied when a substance contains <10⁴ bacteria/ml.

As used herein, the term “bacterial infection” refers to the invasion and colonization of bacteria in a bodily tissue producing subsequent tissue injury and disease.

As used herein, the term “bacterial superinfection” refers to a secondary infection which occurs after a previous infection; this secondary infection is generally more destructive than the first and is often attributed to bacteria which have become resistant to the antibiotics used to treat the first infection. The compositions of the instant invention reduce bacterial infections (and/or superinfections) and prevent further infection from developing.

As used herein, the abbreviation “CFU” refers to colony forming units, a measurement used in order to determine the amount of viable bacteria present.

As used herein, the term “planktonic growth” refers to the growth of bacterial organisms suspended in liquid media in which they move freely.

As used herein, the term “biofilm” refers to the aggregation of bacteria growing upon solid surfaces.

As used herein, the term “weak acid” refers to an acid which undergoes incomplete ionization in water; at one point in time most of the acid occurs in the form of un-ionized molecules. An “organic” acid refers to an acid containing carbon atoms, usually chains of carbon. Vinegar is an impure form of the weak organic acid, acetic acid.

As used herein, the acronym “EDTA” refers to ethylenediaminetetraacetic acid, a metal chelating agent.

As used herein, the term “pH” refers to a measurement of the concentration of hydrogen ions in a solution.

As used herein, the term “synergism” refers to at least two substances working together to increase the total effect, the combination is more effective than either substance alone.

As used herein, the term “placebo” refers to an intentionally ineffective medical treatment. Placebos are used clinically to compare results of treatment against no treatment. The experimental treatment must achieve results above the placebo in order to be deemed effective.

As used herein, the phrase “effective amount” refers to the amount of weak acid or acids sufficient to produce a reduction in microbial contamination.

As used herein, the phrase “pharmacologically effective carrier” refers to any carrier approved for use in humans and animals which facilitates delivery of the weak acids of the composition of the instant invention without interfering with their therapeutic effect. The carrier of the instant invention is an inert carrier vehicle which exhibits no pharmacologic or therapeutic action.

As used herein, the term “therapeutic” refers to any beneficial result of a treatment, particularly reduction and/or prevention of bacterial infection/superinfection.

As used herein, the abbreviation “AA” refers to acetic acid.

As used herein, the abbreviation “CA” refers to citric acid.

As used herein, the abbreviation “PBS” refers to phosphate-buffered saline.

### BRIEF DESCRIPTION OF THE FIGURES

FIG. 1 is a graph comparing the growth of Pseudomonas aeruginosa in broth containing the composition of the instant invention to growth in broth containing a placebo composition.

FIG. 2 is another graph comparing the growth of Pseudomonas aeruginosa in broth containing the composition of the instant invention to growth in broth containing a placebo composition.

FIG. 3 is a graph comparing the growth of Pseudomonas aeruginosa on three polyurethane sponges; the first sponge containing the composition of the instant invention, the second sponge a placebo composition and the third sponge a well-known clinically-approved antimicrobial agent (5% mafenide acetate. A single application of each composition was applied immediately after seeding the polyurethane sponges with Pseudomonas aeruginosa.

FIG. 4 is a control graph comparing the growth of Pseudomonas aeruginosa on two polyurethane sponges; one sponge containing a placebo composition and the other sponge containing a well-known clinically-approved antimicrobial agent (5% mafenide acetate).

FIG. 5 is a graph comparing the growth of Pseudomonas aeruginosa on three polyurethane sponges; the first containing the placebo composition, the second sponge containing the composition of the instant invention and the third sponge also containing the composition of the instant invention. A single application was applied to all sponges; the composition was removed from the second sponge after 5 minutes and removed from the third sponge after 20 minutes.

FIG. 6 is a graph comparing the growth of Staphylococcus epidermis on three polyurethane sponges; the first containing the placebo composition, the second sponge containing the composition of the instant invention and the third sponge also containing the composition of the instant invention. A single application was applied to all sponges; the composition was removed from the second sponge after 5 minutes and removed from the third sponge after 20 minutes.

FIG. 7 is a graph comparing the growth of Pseudomonas aeruginosa on two polyurethane sponges; one containing a placebo composition and the second containing the composition of the instant invention. A single application of each composition was applied 4 hours after seeding the sponges with bacteria.

FIG. 8 is a graph comparing the growth of Pseudomonas aeruginosa on two polyurethane sponges; one containing a placebo composition and the second containing the composition of the instant invention. Compositions were applied 24 hours after seeding the sponges with bacteria. Three 10 minute applications were applied with the third remaining on the gels for the duration of the experiment.

FIG. 9 is a graph comparing the growth of Pseudomonas aeruginosa on polyurethane sponges; containing either a placebo composition or the composition of the instant invention. A single application of each composition was applied 24 hours after seeding the sponges with bacteria. Compositions were removed after 10 minutes.

### DETAILED DESCRIPTION OF THE INVENTION

The instant invention provides a novel and extremely effective bactericidal composition. This composition is pH-independent and effective against a wide range of both gram-positive and gram-negative bacteria. In contrast to currently available antimicrobial compositions, which require continuous presence at the affected wound area for prolonged periods of time in order to provide any benefit, the composition of the instant invention provides a protective effect even when removed from the affected wound area.

The compositions of the invention contain a carefully controlled acid concentration, typically derived from a weak organic acid compatible with human skin, preferably, an organic acid selected from acetic acid, vinegar, citric acid or combinations thereof. In a preferred embodiment, the composition comprises acetic acid or vinegar in an amount to yield up to about 0.5 to 5% acetic acid, and 2 to 8% citric acid. In a most preferred embodiment, the composition comprises acetic acid or vinegar in an amount equal to up to about 1% acetic acid and 5% citric acid. The composition may be in the form of a liquid, gel, lotion, aerosol, or may be provided in the form of a dressing for application to the skin.

The composition has a pH in the range of about 2.5 to about 4.5. The selection of the pH for the composition is dependent upon the formulation used and the ability of the other components in the formulation to tolerate the acidic pH. The gel formulations based upon CARBOPOL preferably have a pH of about 4.2 which aids in the formulation of the gel.

The composition is formulated as a gel or lotion to provide for longer lasting coverage of the affected areas of the skin. The gel or lotion may be a water based gel using a suitable gelling or thickening agent. Alternatively the lotion may be provided as an emulsion, either an oil in water emulsion or a water in oil emulsion. Such emulsions typically are prepared using conventional ingredients including stiffeners, emollients, emulsifying agents and humectants. Stiffeners are usually oil-soluble fatty alcohols such as stearyl alcohol, cetyl alcohol, lauryl alcohol and myristyl alcohol. Emollients are usually isopropyl myristate, lanolin, lanolin derivatives, isopropyl palmitate, isopropyl stearate and the corresponding sebacates. Emulsifying agents are preferably non-ionic and are usually sorbitan monooleate and polyoxyl 40 stearate. humectants are usually propylene glycol, sorbitol, glycerin and mixtures thereof. The ingredients for the emulsion are selected to be compatible at the desired pH range of about 2.5 to about 4.5. A typical formulation is characterized according to the following where percentages are by weight:

| Component | % by weight |
|---|---|
| Petrolatum | 0-25 |
| Stiffener | 7-45 |
| Emollient | 0-15 |
| Emulsifying agent | 4-16 |
| Humectant | 7-40 |
| Weak Organic acid | 5 |
| Water q.s. | 100 |

The compositions of the present invention are preferably formulated as a water soluble gel which provides sustained concentrations of weak acid. The gel formulation, for example, can utilize CARBOPOL as the gelling agent. CARBOPOL is a common gelling agent in foods, cosmetics, prescription and OTC drugs, is highly hydrophilic and rapidly removed under running water. This allows for ease of re-application to prolong the antibacterial effects of the composition.

CARBOPOL polymers are available from the B.F. Goodrich Company and are high molecular weight, crosslinked, acrylic acid-based polymers. CARBOPOL homopolymers are polymers of acrylic acid crosslinked with allyl sucrose or allylpentaerythritol. CARBOPOL copolymers are polymers of acrylic acid, modified by long chain (C10-C30) alkyl acrylates, and crosslinked with allylpentaerythritol. The resins are generally available as fluffy, white, dry powders (100% effective). The carboxyl groups provided by the acrylic acid backbone of the polymer are responsible for many of the product benefits. CARBOPOL resins have an average equivalent weight of 76 per carboxyl group.
Appearance: fluffy, white, mildly acidic powder
Bulk density: approximately 208 kg/m 3 (13 lbs ft 3 )¹
Specific gravity: 1.41
Moisture content as shipped: 2.0% maximum
Equilibrium moisture content: 8-10% (at 50% relative humidity)
pKa: 6.0±0.5
pH of 1.0% water dispersion: 2.5-3.0
pH of 0.5% water dispersion: 2.7-3.5
Equivalent weight: 76±4
Ash content: 0.009 ppm (average)²
Glass transition temperature: 100-105° C. (212-221° F.)
   Polymers produced in cosolvent (a cyclohexane/ethyl acetate mixture) have a bulk density of 176 kg/m³ (11 lbs/ft³).
   Polymers produced in ethyl acetate have an ash content (as potassium sulfate) of 1-3% on average.

It will be apparent to those skilled in the art that other known gelling agents may be utilized in addition to or in place of CARBOPOL, provided they are effective to form a gel in the desired pH range and do not affect the usefulness of the organic acid.

The compositions of the present invention may also be provided as an aerosol, preferably in a pump container to provide a suitable mist spray for application to the affected area. Such aerosol may be simply an aqueous solution of the weak organic acid or may include other ingredients typically provided in aerosols such as stiffeners, humectants, or herb extracts such as aloe vera so long as the additional ingredients do not affect the bactericidal properties of the composition. An advantage of the aerosol form is that it can be applied to the affected area of the skin without requiring direct physical contact with the skin.

The compositions of the present invention may also be provided in the form of a dressing for application to the skin. The dressing can be a gauze, or other suitable sorbent material which is saturated with the composition of the present invention. The use of the dressing provides a physical barrier aiding in protection of the affected area from potentially abrading contact.

The compositions of the present invention may also be applied in the form of an absorbed gel which is maintained within the fibrous matrix of a sorbent article, e.g. a feminine hygiene product such as a pad or tampon, or alternatively a sorbent diaper or the like. While not wishing to be bound to a particular theory of operation, it is believed that as moisture is sorbed from the body, a pathway is provided for communication of the effective ingredients of the inventive composition with the associated dermal areas of contact.

The embodiments of this invention may be formulated and provided in a kit format, comprising a composition for reducing and/or preventing bacterial infection/superinfection including a therapeutically effective amount of at least two weak organic acids selected from the group including acetic acid, vinegar, citric acid or combinations thereof in a pharmacologically effective carrier in the presence of EDTA, wherein the pH of the composition ranges from 2.5 to 4.5, packaged along with wrapping, bandage, or sorbent personal care/hygiene materials or the like, and instructions for their use.

It is within the purview of the instant invention to include within said kit additional substances that assist in occluding the damaged tissue from the environment, along with other substances that assist in the treatment of the skin. Applicators that assist in delivering the composition may also be included in the kit.

Several embodiments of the formulation of the composition of the instant invention will now be described. It is to be appreciated that the invention is not limited to the specific examples which are merely illustrative of the preferred embodiments.

### Preparation of Acid Gels

A. Two Gels Containing the Following Acids were Made:

1: 1% acetic acid (AA)/5% citric acid (CA) gel (200 mL)

2: 1% acetic acid/4% citric acid gel (200 mL)

B. The Following Solutions were Made According to the Above Acid Concentration Specification Containing 0.1 % EDTA
5% CA+1% AA 4% CA+1% AA

| | | | | |
|---|---|---|---|---|
| CA monohydrate | 11.0 | g | 8.75 | g |
| (Fw. 210) | | | | |
| EDTA 120 g | 0.20 | g | 0.20 | g |
| Distilled Water | to 160 | mL | to 160 | mL |
| Vinegar (˜5% acetic acid) | 40 | mL | 40 | mL |

C. Gel productions in beakers of approximately 300 ml

Magnetic Stirring at Highest Setting (2500 rpm)

3.6 g CARBOPOL 940 NF (1.8 g/100 ml) was added very slowly to the each of the above solutions through a fine metal mesh, avoiding clumps.

The suspension was stirred until smooth and without white CARBOPOL flakes; if a few flakes remain, they are dispersed with a spatula.

10N NaOH 1 ml/0.5 ml/0.2 ml was added at a time to obtain pH 4.2 5% CA+1% AA: 10.3 mL of 10 N NaOH (5.2 mL NaOH/100 mL gel) 4% CA+1% AA: 9.0 mL of 10 N NaOH (4.5 mL NaOH/100 mL gel); gelling of CARBOPOL will stop magnetic stirrer action, remaining stirring is accomplished with spatula.

Gel was transferred to 15 mL and 50 mL Falcon tubes and spun at 5 min/2000 rpm (550 g) to degas the gel.

To avoid evaporation of acetic acid from vinegar it is important not to use vacuum to degas the mixture.

### Other Gel Formations

Acetic acid and citric acid were each made to 5% (wt/v) in distilled H₂O from glacial acetic acid (99.8%) and solid citric acid monohydrate (analytical grade), respectively. Mixtures comprising 4 or 5% citric acid and 1% acetic acid were also made from citric acid monohydrate and commercial, food-grade vinegar containing 5% acetic acid. The mixtures were incubated and stirred at room temperature for 20 minutes, and cellulose filtered, followed by the addition of EDTA to 0.1%. Subsequently, solid CARBOPOL 940 NF or 980 NF was applied slowly to 1.8% while being stirred at high magnetic setting until the suspension lost granularity. 10N NaOH was then slowly stirred in to adjust pH of the mixtures to 4.2 for their gelling. For examples, for every 100 mL of the vinegar and 5% acetic acid gels, 2.8 mL of the alkaline solution was required, whereas for the 5% citric acid gel, 4.3 mL was required.

The actual requirement for 10N NaOH may vary slightly, depending on the specific pH parameter of the distilled water used. Finally, excess air was removed from the gel suspensions by low speed (2000 rpm) centrifugation at room temperature for 5 minutes.

As a control to the acid gels, a neutral (pH 7.0) gel was similarly made from distilled H₂O, EDTA and CARBOPOL 940 NF or 980 NF. Since CARBOPOL mediated gelling is much more efficient at near neutral pH, significantly less CARBOPOL was used for the neutral gel. In addition, a neutral gel was also made from 5% acetic acid titrated with 10N NaOH.

These formulations are summarized below:

Preparation I: Vinegar Formulation

| Ingredient | Amount | |
|---|---|---|
| Vinegar | 100 | mL |
| EDTA | 0.1 | g |
| CARBOPOL | 1.8 | g |
| 10 N NaOH | 2.8 | mL |

Preparation II: 5% Acetic Acid Formulation

| Ingredient | Amount | |
|---|---|---|
| glacial acetic acid (99.8%) | 5.01 | mL |
| Distilled H₂O | 94.99 | mL |
| EDTA | 0.1 | g |
| CARBOPOL | 1.8 | g |
| 10 N NaOH | 2.8 | mL |

Preparation III: 5% Citric Acid Formulation

| Ingredient | Amount | |
|---|---|---|
| Citric acid monohydrate | 5.5 | g |
| Distilled H₂O | to 100 | mL |
| EDTA | 0.1 | g |
| CARBOPOL | 1.8 | g |
| 10 N NaOH | 4.3 | mL |

Preparation IV: 5% Citric Acid+Vinegar Formulation

| Ingredient | Amount | |
|---|---|---|
| Citric acid monohydrate | 5.5 | g |
| Distilled H₂O | to 80 | mL |
| Vinegar | 20 | mL |
| EDTA | 0.1 | g |
| CARBOPOL | 1.8 | g |
| 10 N NaOH | 5.2 | mL |

Preparation V: 4% Citric Acid+Vinegar Formulation

| Ingredient | Amount | |
|---|---|---|
| Citric acid monohydrate | 4.4 | g |
| Distilled H₂O | to 80 | mL |
| Vinegar | 20 | mL |
| EDTA | 0.1 | g |
| CARBOPOL | 1.8 | g |
| 10 N NaOH | 4.5 | mL |

Preparation VI: Distilled H₂O Formulation at pH 7.0

| Ingredient | Amount | |
|---|---|---|
| Distilled H₂O | 600 | mL |
| EDTA | 0.1 | g |
| CARBOPOL | 1.8 | g |
| 10 N NaOH | 1.9 | mL |

Preparation VII: Neutral Formulation (pH 7.0) from 5% Acetic Acid and NaOH

| Ingredient | Amount | |
|---|---|---|
| glacial acetic acid (99.8%) | 5.01 | mL |
| Distilled H₂O | 94.99 | mL |
| EDTA | 0.1 | g |
| CARBOPOL | 1.8 | g |
| 10 N NaOH | 11.0 | mL |

Representative examples of experiments conducted to test the bactericidal efficacy of the compositions described herein follow.

### EXAMPLE 1

Pseudomonas aeruginosa (ATCC 27317, gram negative) was grown at 37° C. in Tryptic Soy broth in a shaking water bath to obtain a log-phase growth culture. The suspension was then washed twice in sterile phosphate-buffered saline (PBS) and re-suspended in sterile PBS. Serial dilutions on Tryptic Soy agar enriched with 5% sheep blood were plated to assess bacterial concentration in the washed inocula.

In order to assess planktonic growth, 200 μl aliquots of either a placebo composition or the composition of the instant invention were added to Tryptic Soy broth containing 10⁴ Colony Forming Units (CFU)/ml. The bacterial mixtures were manually mixed until all gel appeared dissolved, and then the bacterial mixtures were incubated at 37° C. for 48 hours. Bacterial growth was assessed at various intervals during the incubation period using well-known microbiological procedures.

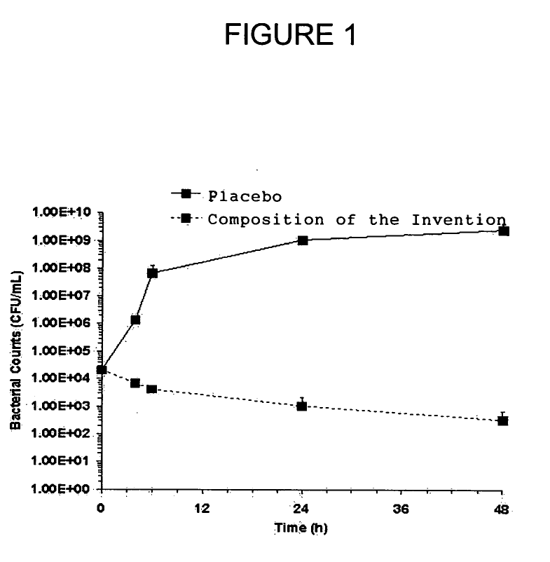

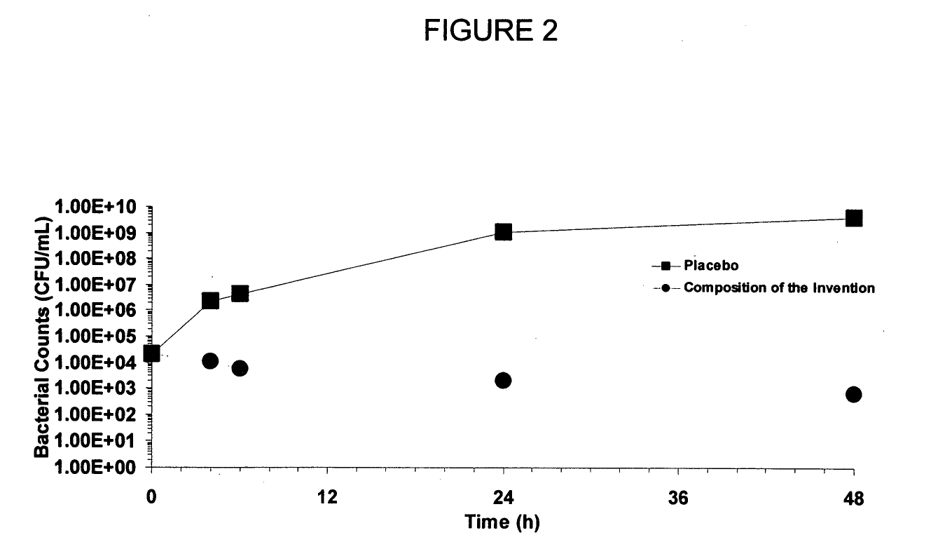

A 2-log reduction in Pseudomonas aeruginosa counts was observed within 3 hours of incubation of the broth containing the composition of the instant invention as compared with the broth containing the placebo composition. While planktonic growth plateaued for the 48-hour experiment using the composition of the instant invention (3.3×10² CFU/ml) a 7-log increase was observed with the placebo composition. FIGS. 1 and 2 show graphs illustrating the data from this experiment. Data are expressed as means±SEM (n=6).

### EXAMPLE 2

Pseudomonas aeruginosa (ATCC 27317) was cultured as in Example 1.

This study emulated a situation wherein a wound is superficially infected and immediately treated.

In order to assess the ability of the composition of the instant invention to eradicate bacterial biofilms, an in vitro polyurethane sponge model was used to stimulate both superficially and deeply-infected wounds. The polyurethane sponges were placed in shallow trays of water and seeded with 10² CFU of Pseudomonas aeruginosa. 200 μl aliquots of a placebo composition, a placebo composition with 5% mafenide acetate (a well-known clinically approved antiseptic gel) and the composition of the instant invention were each applied to a polyurethane sponge immediately after the sponge was seeded with bacteria. The compositions were left on the sponges for a 72 hour period. Bacterial growth was assessed at various time intervals during incubation at 37° C. for the 72 hour period.

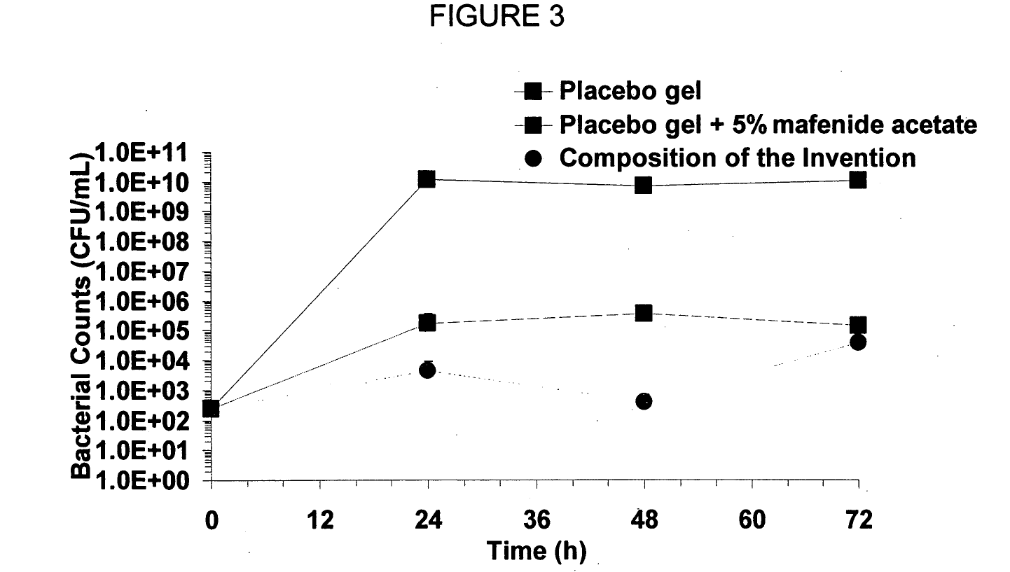

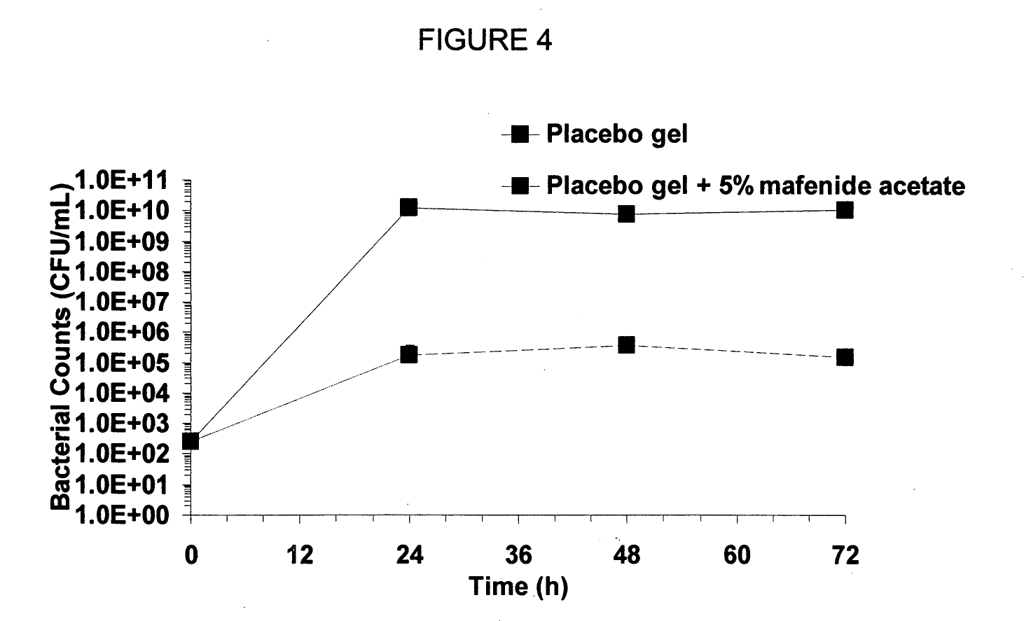

Bacterial counts in the sponges coated with the placebo composition increased to 10¹⁰ CFU within 24 hours and the levels plateaued for the next 48 hours. In contrast, a single application of the composition of the instant invention maintained the bacterial counts below 10⁴ CFU for 72 hours. Furthermore, the bacterial counts in the sponges were up to 3-log lower for the first 48 hours when applying the composition of the instant invention than when using the clinically-approved antiseptic composition. FIG. 3 shows a graph illustrating data from this experiment. FIG. 4 is a control graph comparing the growth of Pseudomonas aeruginosa on two polyurethane sponges; one sponge containing a placebo composition and the other sponge containing a well-known clinically-approved antimicrobial agent(5% mafenide acetate). Data are expressed as means±SEM (n=6).

### EXAMPLE 3

Pseudomonas aeruginosa (ATCC 27317) was cultured as in Example 1.

This experiment was conducted in order to determine the minimum duration of the composition application that would exert a significant bactericidal effect. Four polyurethane sponges were placed in a shallow tray of water and seeded with 10³ CFU of Pseudomonas aeruginosa. A 200 μl aliquot (single application) of a placebo composition was applied to sponges 1 and 2 and a 200 μl aliquot of the composition of the instant invention was applied to sponges 3 and 4. Aliquots were placed on the sponges immediately after bacterial seeding. The compositions were removed from sponges 1 and 3 after 5 minutes and removed from sponges 2 and 4 after 20 minutes. The sponges were then incubated at 37° C. for a period of 72 hours.

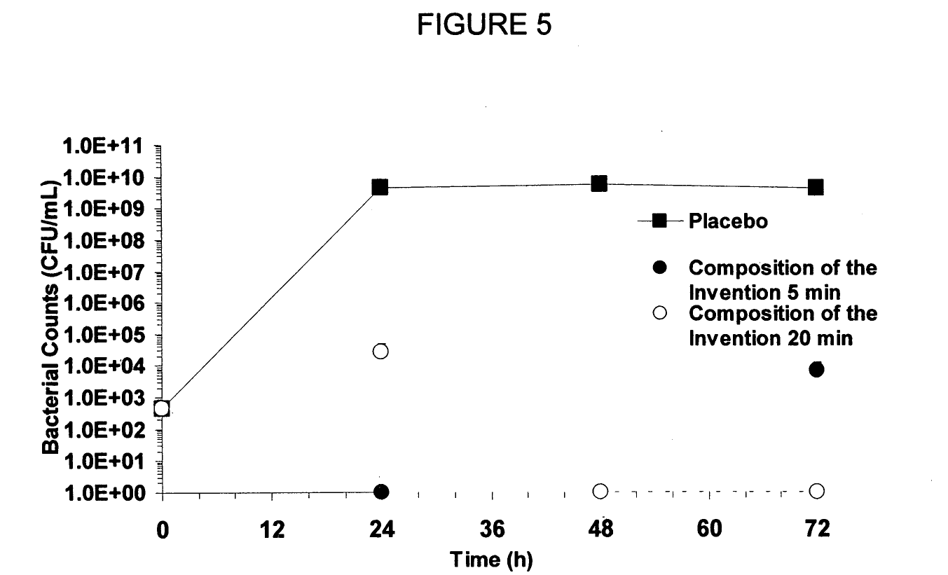

Bacterial counts in the sponges coated with the placebo composition increased to at least 10⁹ CFU within 24 hours; the levels plateaued for the next 48 hours. In contrast, a single 5 minute application of the composition of the instant invention eradicated Pseudomonas aeruginosa for 48 hours after seeding. Increasing the duration of application of the composition of the instant invention to 20 minutes reduced the Pseudomonas aeruginosa counts by 5-log within 24 hours, with no growth being observed for the remainder of the study. FIG. 5 shows a graph illustrating data from this experiment. Data are expressed as means±SEM (n=6).

### EXAMPLE 4

This experiment repeated the experiment in Example 3 using Staphylococcus epidermis (ATCC 12228, gram positive) in place of Pseudomonas aeruginosa. Staphylococcus epidermis was cultured in the same manner as Pseudomonas aeruginosa in Example 1.

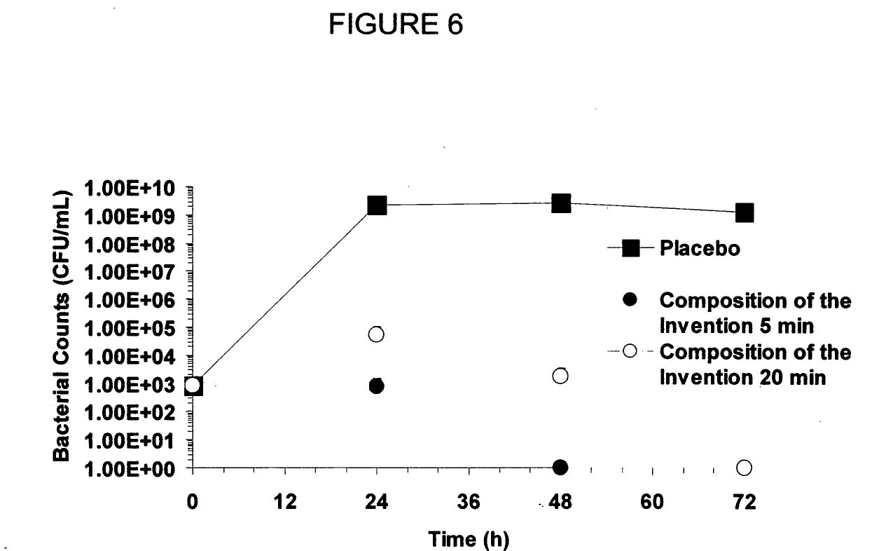

A single 5 minute application of the composition of the instant invention eradicated Staphylococcus epidermis after 48 hours. FIG. 6 shows a graph illustrating data from this experiment. Data are expressed as means±SEM (n=6).

### EXAMPLE 5

Pseudomonas aeruginosa (ATCC 27317) was cultured as in Example 1.

This experiment emulates a scenario wherein treatment is delayed, thus the wound becomes increasingly infected before any medical intervention.

Two polyurethane sponges were placed in a shallow tray of water and seeded with 10³ CFU of Pseudomonas aeruginosa. A 200 μl aliquot of a placebo composition was applied to the first sponge 4 hours after bacterial seeding and a 200 μl aliquot of the composition of the instant invention was applied to the second sponge 4 hours after bacterial seeding. The sponges were then incubated at 37° C. for 72 hours with the compositions remaining in place for the duration of the experiment. Bacterial growth was assessed at various time intervals during the 72 hour period.

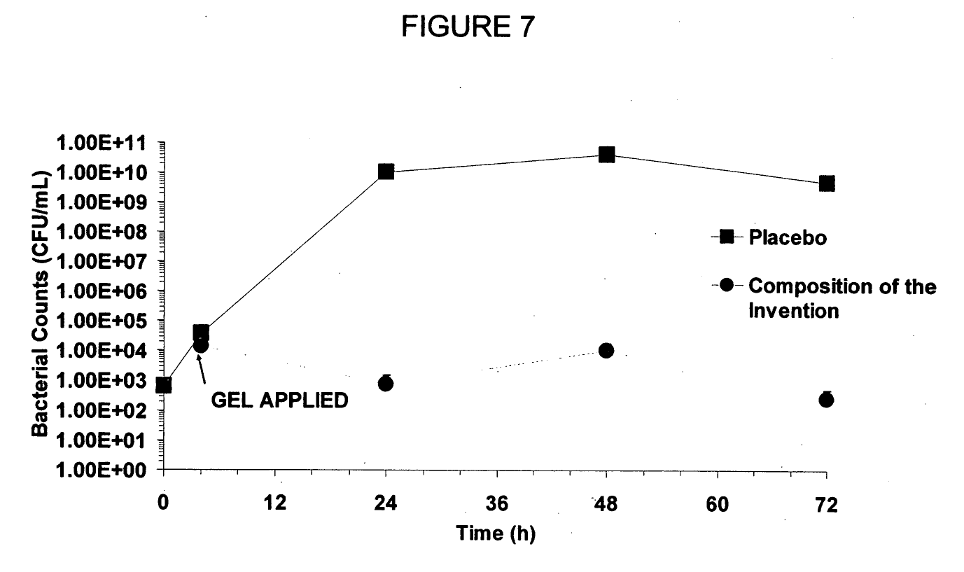

Pseudomonas aeruginosa levels increased by one-log within 4 hours of seeding the sponges. Bacterial counts in the sponge coated with the placebo composition increased to 10¹⁰ CFU within 24 hours. The composition of the instant invention maintained the bacterial count below 10⁴ CFU for 72 hours. FIG. 7 shows a graph illustrating data from this experiment. Data are expressed as means±SEM (n=6).

### EXAMPLE 6

Pseudomonas aeruginosa (ATCC 27317) was cultured as in Example 1.

This experiment emulates a scenario wherein treatment is markedly delayed, thus the wound becomes increasingly infected before any medical intervention.

Two polyurethane sponges were placed in a shallow tray of water and seeded with 10³ CFU of Pseudomonas aeruginosa. A 200 μl aliquot of a placebo composition was applied to the first sponge 24 hours after bacterial seeding and a 200 μl aliquot of the composition of the instant invention was applied to the second sponge 24 hours after bacterial seeding. The first aliquot remained on the sponges for 10 minutes after application, was removed and replaced by a second application. The second application remained on the sponges for 10 minutes after application, was removed and replaced with a third application which remained on the sponges for the duration of the experiment. The sponges were incubated at 37° C. for 96 hours. Bacterial growth was assessed at various time intervals during the 96 hour period.

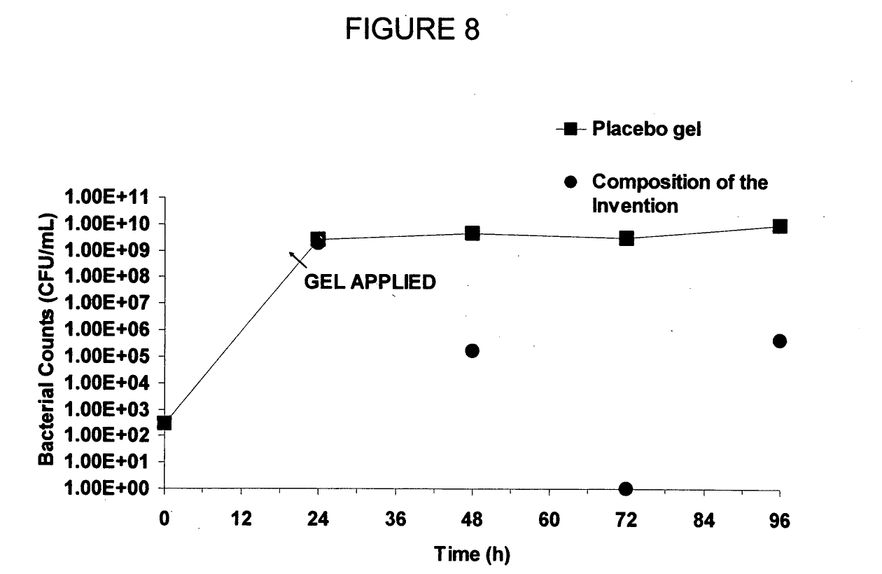

Pseudomonas aeruginosa levels increased to 10⁹ CFU within 24 hours of bacterial seeding. Bacterial counts in the sponge coated with the placebo composition remained constant. In contrast, a 4-log reduction in bacteria was observed within 24 hours of the application of the composition of the instant invention. No bacteria were observed in the sponge 48 hours after the application of the composition of the instant invention, however bacterial counts increased to 10⁵ CFU within 72 hours. FIG. 8 shows a graph illustrating data from this experiment. Data are expressed as means±SEM (n=6).

### EXAMPLE 7

Pseudomonas aeruginosa (ATCC 27317) was cultured as in Example 1.

This experiment emulates a scenario wherein treatment is markedly delayed, thus the wound becomes increasingly infected before any medical intervention.

Six polyurethane sponges were placed in a shallow tray of water and seeded with 10³ CFU of Pseudomonas aeruginosa. A 200 μl aliquot of a placebo composition was applied to the first three sponges 24 hours after bacterial seeding and a 200 μl aliquot of the composition of the instant invention was applied to the last three sponges 24 hours after bacterial seeding. The compositions were removed from the sponges 10 minutes after application. The sponges were incubated at 37° C. for 96 hours. Bacterial growth was assessed at various time intervals during the 96 hour period.

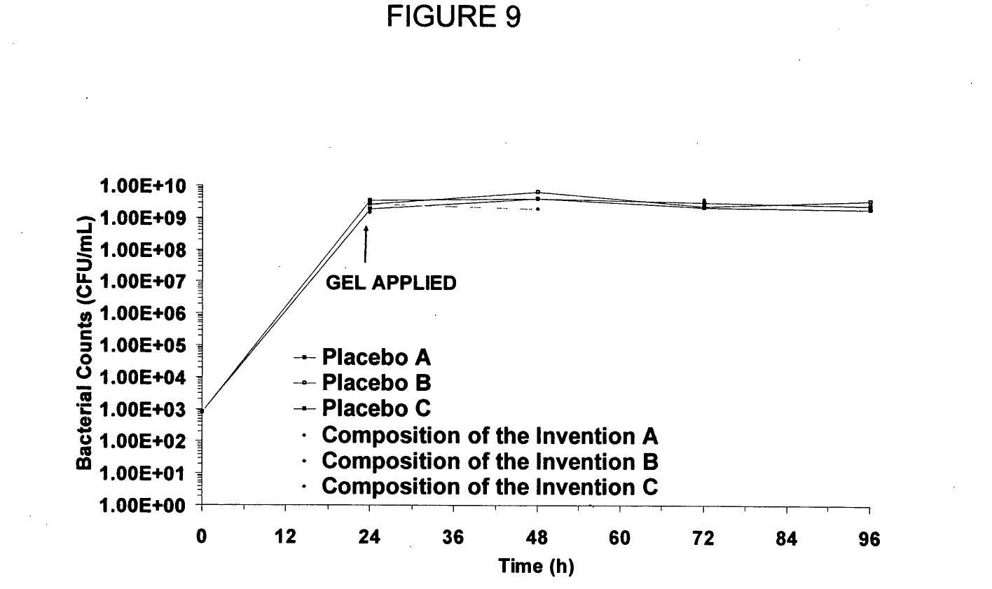

Pseudomonas aeruginosa levels increased to 10¹⁰ CFU within 24 hours of seeding all sponges. Bacterial counts in the sponges that had been coated with the placebo composition remained constant for the remainder of the experiment. In contrast, a 3-log reduction was observed within 24 hours of application of the composition of the instant invention, this bactericidal effect thereafter maintained for up to 96 hours. FIG. 9 shows a graph illustrating data from this experiment. Data are expressed as means±SEM (n=6).

The data disclosed herein strongly suggest the efficacy of the composition of the instant invention in drastically reducing both planktonic and biofilm bacterial growth. This bactericidal effect was observed whether the composition was applied immediately upon infection or after a delay of up to 24 hours.

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The compositions, related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A method for reducing and/or preventing bacterial infection/superinfection of a wound comprising applying to an affected area a composition including a therapeutically effective amount of at least two weak organic acids selected from the group consisting of acetic acid, vinegar, citric acid and combinations thereof in a pharmacologically effective carrier, in the presence of ethylene diamine tetraacetic acid (EDTA), wherein the pH of said composition ranges from approximately 2.5 to 4.5; and wherein said therapeutically effective amount provides reduction and/or prevention of bacterial infection/superinfection of said wound.

2. The method in accordance with claim 1 wherein said applying occurs immediately after exposure of said wound to the external environment.

3. The method in accordance with claim 1 wherein said applying is delayed for a period of time after exposure of said wound to the external environment.

4. The method in accordance with claim 3 wherein said applying is delayed for 4 hours after exposure of said wound to the external environment.

5. The method in accordance with claim 3 wherein said applying is delayed for 24 hours after exposure of said wound to the external environment.

6. The method in accordance with claim 1 wherein said composition is removed from said affected area during a period of time between 5 and 20 minutes after said applying.

7. The method in accordance with claim 6 wherein said composition is removed from said affected area 5 minutes after said applying.

8. The method in accordance with claim 6 wherein said composition is removed from said affected area 10 minutes after said applying.

9. The method in accordance with claim 6 wherein said composition is removed from said affected area 20 minutes after said applying.

10. The method in accordance with claim 1 wherein the pH of said composition ranges from 4.1 to 4.4.

11. The method in accordance with claim 1 wherein said pharmacologically effective carrier includes a gelling agent.

12. The method in accordance with claim 11 wherein said gelling agent is a cross-linked homopolymer or co-polymer of acrylic acid.

13. A kit for reducing and/or preventing bacterial infection/superinfection of wounds comprising:
a) a composition for reducing and/or preventing bacterial infection/superinfection of a wound including a therapeutically effective amount of at least two weak organic acids selected from the group consisting of acetic acid, vinegar, citric acid and combinations thereof in a pharmacologically effective carrier in the presence of EDTA, and wherein the pH of said composition ranges from about 2.5 to 4.5;
b) wrapping or bandage materials selected from the group consisting of wound dressings, absorbent bandages, feminine hygiene products, and diapers; and
c) instructions for use.
